(19) United States
(12) Patent Application Publication
(10) Pub. No.: US 2020/0243173 A1
(43) Pub. Date: Jul. 30, 2020

(54) PATIENT MANAGEMENT APPARATUS AND PATIENT MANAGEMENT METHOD

(71) Applicant: NEC CORPORATION, Tokyo (JP)

(72) Inventors: Takafumi Yoshida, Tokyo (JP); Keisuke Nozawa, Tokyo (JP); Masahiro Komai, Tokyo (JP); Daisuke Fukai, Tokyo (JP); Misa Tsuchida, Tokyo (JP)

(73) Assignee: NEC CORPORATION, Tokyo (JP)

(21) Appl. No.: 16/773,373

(22) Filed: Jan. 27, 2020

(30) Foreign Application Priority Data
Jan. 30, 2019 (JP) 2019-014611

(51) Int. Cl. G16H 10/60; G06Q 10/06; G06Q 50/22; G06Q 10/10

(52) U.S. Cl. CPC G16H 10/60; G06Q 10/10; G06Q 50/22; G06Q 10/06311

(57) ABSTRACT

A patient management apparatus includes an electronic medical record part configured to record execution results of medical examinations for patients in connection with identifications of patients, acknowledge flags indicating whether the execution results have been acknowledged, and explanatory flags indicating whether the execution results have been explained to patients, and a report management part configured to determine patients to be examined according to a today's medical examination schedule in connection with the identifications of the patients and to thereby output a list of reading requests to read the execution results, the acknowledge flags, and the explanatory flags in connection with the identifications of the patients to be examined according to the today's medical examination schedule.

### CROSS-REFERENCE TO RELATED APPLICATION

The present application claims the priority benefit of Japanese Patent Application No. 2019-14611 filed on Jan. 30, 2019, the subject matter of which is hereby incorporated herein by reference.

### BACKGROUND OF THE INVENTION

### 1. Field of the Invention

The present invention relates to a patient management apparatus and a patient management method.

### 2. Description of Related Art

In medical facilities, doctors need to confirm many results of medical examination performed on patients in each day. As types of medical examination, for example, it is possible to mention radiation photography, endoscopic examination, phathological examination, specimen examination, and bacteriological examination. After performing medical examination on patients, doctors may need to explain conditions of patients upon confirming results of medical examination. Doctors may need to make a new medical explanation about new items of medical examination on patients in consideration of a previous medical explanation made in a diagnostic stage in the past. Patent Document 1 discloses a diagnostic explanation support device configured to support doctors to provide patients with various items of medical explanation including previous and new items of medical examination. Patent Document 2 discloses a data output device configured to output time-series data about conditions of patients such as vital signs and results of specimen examination on a computer screen.

### 3. Patent Documents

Patent Document 1: Japanese Patent Application Publication No. 2009-211289

Patent Document 2: Japanese Patent Application Publication No. 2015-197736

### 4. Technical Problem

To achieve the accountability of doctors to patients, it is required for doctors to explain conditions of patients without overlooking results of medical examination. In this connection, Patent Document 1 and Patent Document 2 can simply provide doctors with a medical presentation about conditions of patients. However, it is difficult for doctors to explain all the necessary matters about conditions of patients using such a simple medical presentation without making any errors. For this reason, it is required for medical facilities to improve the accuracy of explanation about conditions of patients based on results of medical examinations in communication with patients.

The present invention is made in consideration of the aforementioned circumstances, and therefore the present invention aims to provide a patient management apparatus and a patient management method, which can solve the above problem.

### SUMMARY OF THE INVENTION

In a first aspect of the invention, a patient management apparatus includes an electronic medical record part configured to acquire and record the execution results of medical examinations for patients in connection with the identifications of patients, acknowledge flags indicating whether the execution results have been acknowledged, and explanatory flags indicating whether the execution results have been explained to patients, and a report management part configured to determine patients to be examined according to a today's medical examination schedule in connection with the identifications of the patients and to thereby output a list of reading requests to read the execution results, the acknowledge flags, and the explanatory flags in connection with the identifications of the patients to be examined according to the today's medical examination schedule.

In a second aspect of the invention, a patient management method includes the steps of: acquiring and recording the execution results of medical examinations for patients in connection with the identifications of patients, acknowledge flags indicating whether the execution results have been acknowledged, and explanatory flags indicating whether the execution results have been explained to patients; determining patients to be examined according to a today's medical examination schedule in connection with the identifications of the patients; and outputting a list of reading requests to read the execution results, the acknowledge flags, and the explanatory flags in connection with the identifications of the patients to be examined according to the today's medical examination schedule.

According to the present invention, it is possible to improve an accuracy of explaining to patients their conditions based on the execution results of medical examinations for patients. In addition, it is possible to manage the status as to whether or not patients have an explanation for the execution results of medical examinations.

### BRIEF DESCRIPTION OF THE DRAWINGS

FIG. 1 is a schematic diagram of a patient management system including a patient management apparatus and an examination management apparatus.

FIG. 2 is a block diagram showing the hardware configuration applicable to the patient management apparatus and the verification management apparatus.

FIG. 3 is a block diagram showing the functional configuration of the patient management apparatus according to the embodiment of the present invention.

FIG. 4 shows an example of a report management table produced by the patient management apparatus according to the embodiment of the present invention.

FIG. 5 is a flowchart showing the process of the patient management apparatus according to the embodiment of the present invention.

FIG. 6 shows an example of a report management page produced using the report management table.

FIG. 7 shows a part of the report management page shown in FIG. 6.

FIG. 8 shows a part of the report management page shown in FIG. 6.

FIG. 9 shows a part of the report management page shown in FIG. 6.

FIG. 10 shows a part of the report management page shown in FIG. 6.

FIG. 11 shows a part of the report management page shown in FIG. 6.

FIG. 12 shows a part of the report management page shown in FIG. 6.

FIG. 13 shows a part of the report management page shown in FIG. 6.

FIG. 14 shows an example of an examination request registration page.

FIG. 15 shows a part of the report management page shown in FIG. 6.

FIG. 16 shows a part of the report management page shown in FIG. 6, which is updated in columns relating to an assessment report.

FIG. 17 is a block diagram showing a minimum configuration of the patient management apparatus.

### DETAILED DESCRIPTION OF THE EMBODIMENT

The present invention will be described in detail by way of the embodiment with reference to the accompanying drawings.

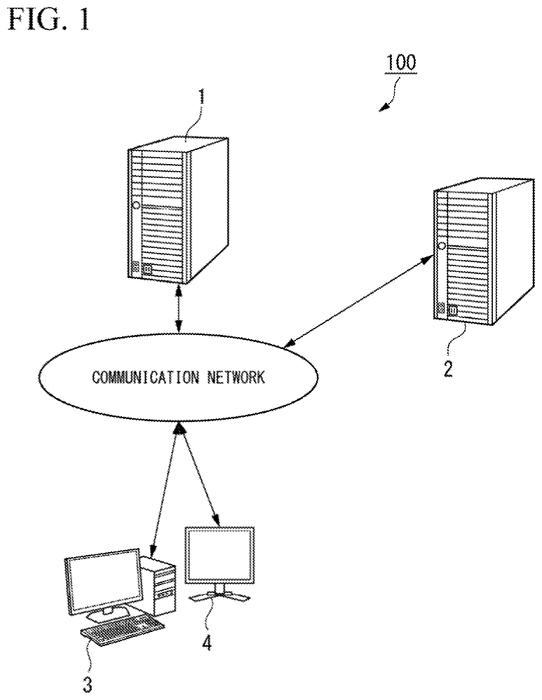

FIG. 1 is a schematic diagram of a patient management system 100 including a patient management apparatus 1, an examination management apparatus 2, a first terminal 3, and a second terminal 4, which are connected together through a communication network. For example, the patient management system 100 is installed in a large-scale hospital having several hundreds of beds for patients. The patient management system 100 may further include other devices in communication with the constituent elements 1-4 shown in FIG. 1.

The patient management apparatus 1 is configured to have doctors confirm results of medical examination, to make an explanation to patients based on results of medical examination, to record confirmation and explanation by doctors, and to further record various comments about patients.

The examination management apparatus 2 is configured to manage results of medical examination with respect to patients. The examination management apparatus 2 may be directly connected to medical examination devices so as to retrieve data generated by medical examination devices. As medical examination devices, it is possible to mention radiotherapy equipment, magnetic resonance imaging equipment, and X-ray imaging equipment.

The first terminal 3 communicates with the patient management apparatus 1 to output the information transmitted by the patient management apparatus 1 with a monitor or the like. The second terminal 4 communicates with the examination management apparatus 2 to output the information transmitted by the examination management apparatus 2 with a monitor or the like. In this connection, it is possible to provide a common terminal sharing the functions of the first terminal 3 and the second terminal 4.

As a medical examination device, the present embodiment uses radiotherapy equipment. As the examination management apparatus 2, the present embodiment uses an apparatus configured to display radiographic images received from the radiotherapy equipment and to thereby manage the information registered by an image-reading doctor (IMAGE-READING DOC), a clinical technician, or the like.

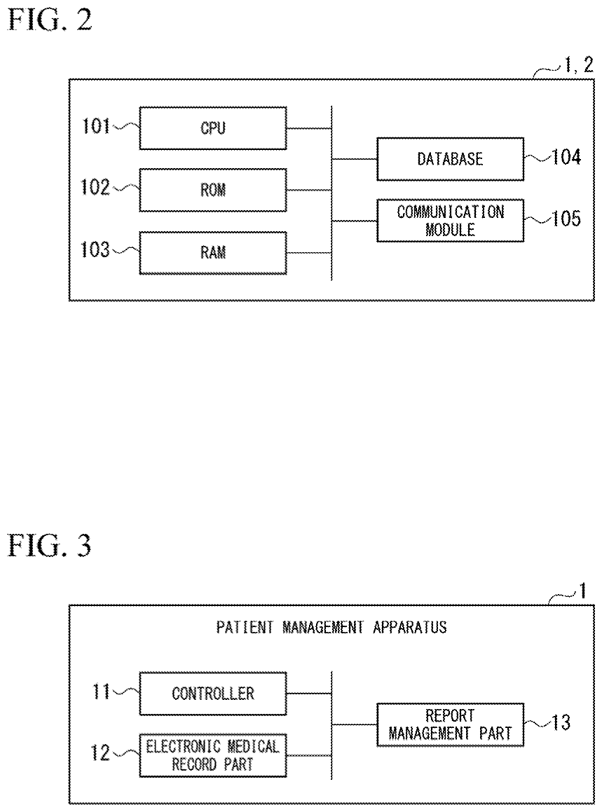

FIG. 2 is a block diagram showing the hardware configuration applicable to the constituent elements 1-4, i.e. the patient management apparatus 1, the examination management apparatus 2, the first terminal 3, and the second terminal 4. The constituent elements 1-4 are each configured of a computer having various hardware elements such as a CPU (Central Processing Unit) 101, a ROM (Read-Only Memory) 102, a RAM (Random-Access Memory) 103, a database 104, and a communication module 105.

FIG. 3 is a block diagram showing the functional configuration of the patient management apparatus 1. The patient management apparatus 1 is realized by the CPU 101 executing programs (e.g. a patient-management application) registered in the database 104. Accordingly, the patient management apparatus 1 may have various functional parts such as a controller 11, an electronic medical record part 12, and a report management part 13.

The controller 11 is configured to control other functional parts. The electronic medical record part 12 is configured to electronically record and output the electronic medical record information for each patient. The electronic medical record information includes the record information such as names of patients, patient numbers, impatient wards, room numbers, and results of medical examination for patients. The record information may further include SOAP types and record contents. Herein, the term “SOAP” is made up of initial characters selected from “Subjective” (i.e. subjective information), “Objective” (i.e. objective information), “Assessment” (i.e. assessment information), and “Plan” (i.e. planning or therapeutic information); hence, the SOAP represents types of those pieces of information. In addition, symbols S, O, A, and P are used to represent Subjective type, Objective type, Assessment type, and Planning type respectively. Specifically, Subjective type (S) represents the information directly obtained from a patient while Objective type (O) represents the information obtained by a nurse making a physical examination of a patient. In addition, Assessment (A) represents the information produced by the assessment made by a nurse or a doctor based on Subjective (S) and Objective (O). Plan (P) represents planning or therapy to be made as a result of Assessment (A). Users such as doctors may register the record content for each SOAP type in the patient management system 100 as the information to be recorded as an electronic medical record for each patient. The present embodiment refers to the patient management apparatus 1 having the function of the electronic medical record part 12, whereas the function of the electronic medical record part 12 can be installed in another apparatus such as an electronic medical record server.

The report management part 13 has the function to complement the process of the electronic medical record part 12. In the present embodiment, the report management part 13 acquires an execution result due to an execution request of a medical examination for a patient from the examination management apparatus 2, and then the report management part 13 records the execution result in connection with an acknowledge (ACK) flag as to whether or not to acknowledge the patient number and the execution result and an explanatory (EXPL) flag as whether or not to explain the execution result. The report management part 13 is configured to determine a patient subjected to a today's medical examination according to a medical examination schedule for each patient number. The report management part 13 is configured to transmit to the first terminal 3 a read-request list of execution results connected to patient numbers of patients other than patients each having an ACK flag (indicating a completion of acknowledgement) and an EXPL flag (indicating a completion of explanation) among all the patients subjected to medical examinations according to the today's medical examination schedule. The detailed process of the report management part 13 will be described later. According to the process of the report management part 13, it is possible to improve an accuracy of explaining conditions of patients based on execution results of medical examinations, and therefore it is possible to manage the status as to whether or not to complete explaining conditions of patients based on execution results of medical examinations.

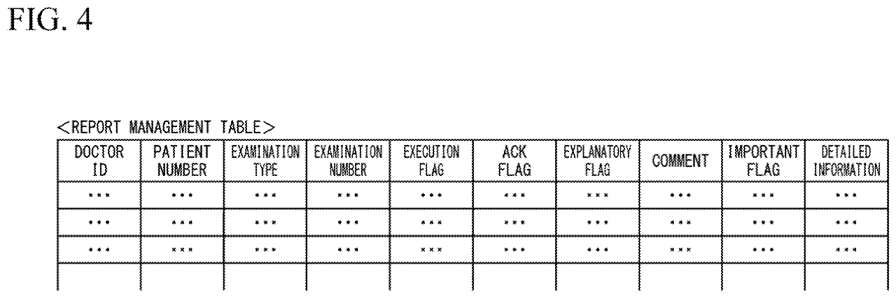

FIG. 4 shows an example of a report management table stored on a database. The report management table registers various pieces information such as DOC ID, PNT NO, EXAM TYPE, EXAM NO, EXC FLAG, ACK FLAG, EXPL FLAG, COMMENT, IMPORTANT FLAG, and DETAILED INFORMATION, which are linked together. Herein, DOC ID (i.e. a doctor ID) is a user identification such as an identification of a doctor; PNT NO (i.e. a patient number) is an identification of a patient examined by a doctor having DOC ID; EXAM TYPE (i.e. an examination type) is an identification of a medical examination made on a patient having PNT NO; EXAM NO (i.e. an examination number) is the number of a medical examination; EXC FLAG (i.e. an execution flag) is a value representing whether or not to complete a medical examination with respect to the information representing an execution result of a medical examination for a patient due to an examination request of a medical examination made by the doctor; ACK FLAG (i.e. an acknowledge flag) is a value representing whether or not the doctor has acknowledged the assessment report corresponding to the execution result; EXPL FLAG (i.e. an explanatory flag) is a value representing whether or not the doctor has completed explaining the assessment report; COMMENT is a comment input by the doctor (or a user); IMPORTANT FLAG is a value representing a degree of importance annotated by the doctor; and DETAILED INFORMATION refers to records such as a patient name (PNT NAME), a version (VERSION) indicating the latest version of an assessment report, the name of a doctor requesting acknowledgement of the assessment report (DOC REQ), the name of a doctor serving as a notice destination of an acknowledge request (NOTICE DESTN) , the date to make a medical examination (EXAM DATE), the date to produce the assessment report (REPORT DATE), the name of a doctor producing the assessment report (DOCUMENT as a documenter), and the department of medical request assigned to the doctor (DEPT REQUEST) as well as a last person to update those records (LAST PERS), and a last date to update those records (LAST DATE).

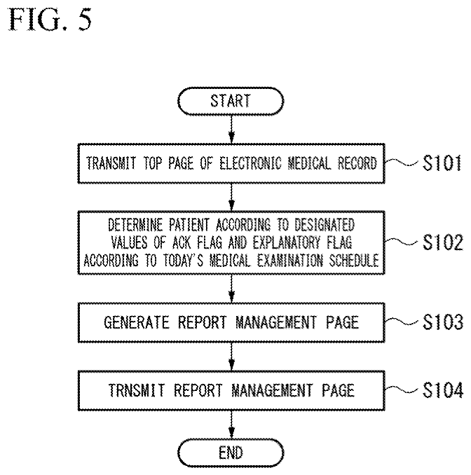

Upon making a medical examination for a patient, the examination management apparatus 2 produces the execution result of a medical examination. The execution result may include the update information to update the execution flag from a non-execution of examination to an execution of examination. The execution result may further include an assessment report which is made by assessing the execution-result data of a medical examination made by an examination device. The examination management apparatus 2 may separately produce and transmit the update information and the assessment report to the patient management apparatus 1. When the examination device is radiotherapy equipment, for example, the examination management apparatus 2 obtains a patient number, an examination number, and radiographic images (representing the execution result of a medical examination) from the radiotherapy equipment. An image-reading doctor produces an assessment report including a comment about the result of reading radiographic images using the examination management apparatus 2. The image-reading doctor may register the data of an assessment report in the examination management apparatus 2 in connection with the patient number and the examination number. Upon detecting registration of the assessment report, the examination management apparatus 2 transmits to the patient management apparatus 1 the information including the patient number relating to a medical examination assessed in the assessment report, the examination number, a completion of registering the assessment report, and the version of the assessment report. The report management part 13 of the patient management apparatus 1 determines the execution flag, the acknowledge flag, the explanatory flag, and the version which are linked to the patient number and the examination number in the report management table (FIG. 5). Upon producing a first version of the assessment report, the report management part 13 updates the execution flag to indicate an execution of examination, updates the acknowledge flag to indicate a negative acknowledge (NAK), and changes the explanatory flag to indicate an incompletion of explanation.

FIG. 5 is a flowchart showing the process of the patient management apparatus 1 (steps S101 to S104). Hereinafter, the processes of the patient management apparatus 1, the examination management apparatus 2, the first terminal 3, and the second terminal 4 will be described in detail.

First, a user (e.g. a doctor) of the patient management system 100 may operate the first terminal 3 to display a top page to be transmitted to the patient management apparatus 1. At this time, the electronic medical record part 12 receives a doctor ID serving as a user (or a doctor) and a password from the first terminal 3 so as to retrieve the information of a patient consulted by the doctor, which will be stored based on the doctor ID and the password. The electronic medical record part 12 transmits to the first terminal 3 the top page of an electronic medical record used to display a list of patient information representing patients consulted by the doctor (S101). After transmitting the top page of an electronic medical record, the electronic medical record part 12 outputs the doctor ID to the report management part 13.

The report management part 13 produces a report management page to be confirmed by the doctor based on the doctor ID. The report management page displays a list of patient information representing patients whom the doctor has not yet confirmed their assessment reports describing execution results of medical examinations and patients whom the doctor has not explained execution results of medical examinations based on their assessment reports among all the patients to be examined according to the today's medical examination schedule.

The report management part 13 produces the report management page using the report management table (FIG. 4), which are produced as described before. Specifically, the report management part 13 acquires from the first terminal 3 acknowledgement flags and explanatory flags which are designated by the user who operates the first terminal 3. Subsequently, the report management part 13 determines patients according to the today's medical examination schedule based on acknowledge flags and explanatory flags which are acquired from the first terminal 3 (S102). Specifically, when the acknowledge flag acquired from the first terminal 3 indicates “unacknowledged”, the report management part 13 determines a patient indicating an unacknowledged status of an assessment report describing the execution result of a medical examination. When the acknowledge flag acquired from the first terminal 3 indicates “acknowledged”, the report management part 13 determines a patient indicating an acknowledged status of an assessment report describing the execution result of a medical examination. When the acknowledge flag acquired from the first terminal 3 indicates neither “unacknowledged” nor “acknowledged”, the report management part 13 determines both a patient indicating an unacknowledged status of an assessment report and a patient indicating an acknowledged status of an assessment report.

When the explanatory flag acquired from the first terminal 3 indicates “an incompletion of explanation”, the report management part 13 determines a patient having no explanation for the execution result of a medical examination based on an assessment report. When the explanatory flag acquired from the first terminal 3 indicates “a completion of explanation”, the report management part 13 determines a patient having an explanation for the execution result of a medical examination based on an assessment report. When the explanatory flag acquired from the first terminal 3 indicates neither “an incompletion of explanation” nor “a completion of explanation”, the report management part 13 determines both a patient having no explanation for the execution result of a medical examination and a patient having an explanation for the execution result of a medical examination.

When the acknowledge flag acquired from the first terminal 3 indicates “unacknowledged” while the explanatory flag acquired from the first terminal 3 indicates “a completion of explanation”, the report management part 13 determines a patient having an unacknowledged status of an assessment report describing the result of a medical examination but having an explanation for the execution result of a medical examination based on an assessment report.

When the acknowledge flag acquired from the first terminal 3 indicates “unacknowledged” while the explanatory flag acquired from the first terminal 3 indicates “an incompletion of explanation”, the report management part 13 determines a patient having an unacknowledged status of an assessment report describing the result of a medical examination and having no explanation for the execution result of a medical examination based on an assessment report.

When the acknowledge flag acquired from the first terminal 3 indicates “acknowledged” while the explanatory flag acquired from the first terminal 3 indicates “a completion of explanation”, the report management part 13 determines a patient having an acknowledged status of an assessment report describing the result of a medical examination and an explanation for the execution result of a medical examination based on an assessment report.

When the acknowledge flag acquired from the first terminal 3 indicates “acknowledged” while the explanatory flag acquired from the first terminal 3 indicates “an incompletion of explanation”, the report management part 13 determines a patient having an acknowledged status of an assessment report describing the execution result of a medical examination but having no explanation for the execution result of a medical examination based on an assessment report.

Upon determining patients to be examined according to the today's medical examination schedule, the report management part 13 may determine the patient numbers assigned to patients other than patients having acknowledge flags indicating “acknowledged” and explanatory flags indicating “a completion of explanation”. The report management part 13 may produce a report management page describing a list of information such as examination types, examination numbers, execution flags, acknowledge flags, explanatory flags, comments, degrees of importance, and patient attributes, which are registered in a report management table in connection with the patient numbers representative of the determined patients (S103). The report management part 13 transmits the report management page to the first terminal 3 (S104).

The first terminal 3 receives the top page of an electronic medical record and the report management page. Subsequently, the first terminal 3 displays the top page of an electronic medical record and the report management page on a monitor. Accordingly, a user (e.g. a doctor) of the first terminal 3 is able to read the top page of an electronic medical record together with the report management table when operating the first terminal 3 to display the top page of an electronic medical record on a monitor. The report management page shows the page information which is produced according to an acknowledge flag and an explanatory flag designated by the user, and therefore the report management page shows a list of patients indicating an acknowledged status of an assessment report describing the execution result of a medical examination and patients having no explanation for the execution result of a medical examination based on an assessment report among patients to be examined according to the today's medical examination report. By outputting the top page of an electronic medical record to a monitor of the first terminal 3, the user of the first terminal 3 is able to immediately confirm a list of patients indicating unacknowledged status of an assessment report describing the execution result of a medical examination and patients having no explanation for the execution result of a medical examination based on an assessment report.

The aforementioned process of the patient management apparatus 1 is an exemplary process to produce the page information according to acknowledge flags and explanatory flags, which are designated by a user (e.g. a doctor), i.e. a list of requests to read acknowledge flags and explanatory flags as well as execution results which are linked to the identifications of patients to be examined according to the today's medical examination schedule. In addition, the aforementioned process of the patient management apparatus 1 is an exemplary process to automatically produce a list of requests to read execution results of medical examinations when initiated by a user operation.

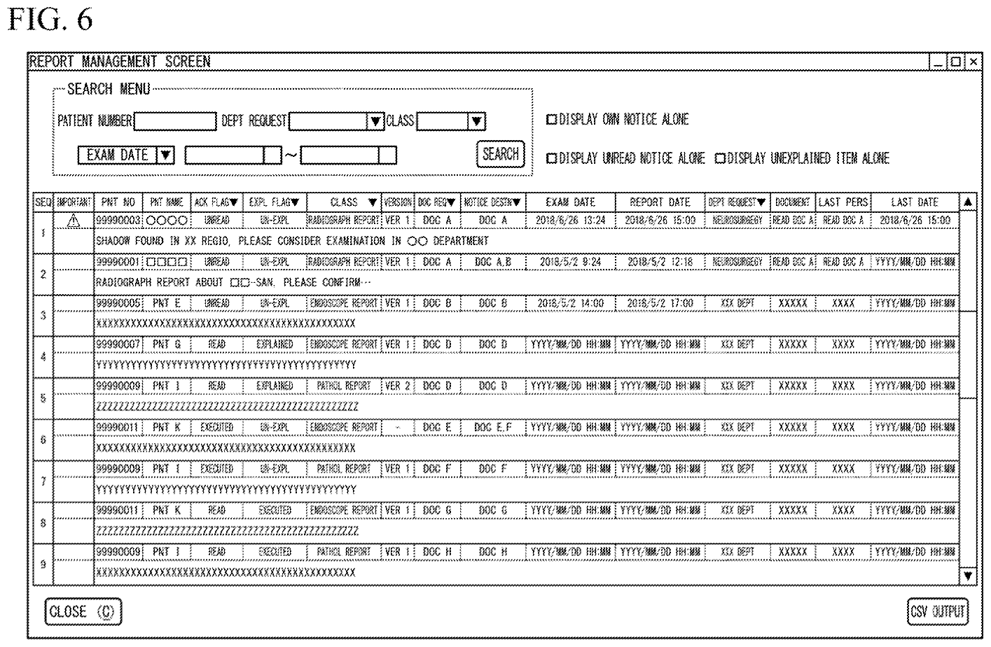

FIG. 6 shows an example of a report management page produced using the report management table. The report management page of FIG. 6 shows an acknowledge flag (ACK FLAG), an explanatory flag (EXPL FLAG), an examination type (EXAM TYPE), a version of an assessment report (VERSION), the name of a doctor making a request (DOC REQUEST), the name of a doctor serving as a notice destination (NOTICE DESTN), the date of a medical examination (EXAM DATE), the date to produce an assessment report (REPORT DATE), the name of a doctor requesting confirmation of an assessment report, a department of medical request assigned to the doctor (DEPT REQUEST), the last person to update records (UPDATE PERS), the last date to update records (UPDAT DATE), a doctor originating a request, and a comment made by a doctor producing an assessment report in connection with a patient number (PNT NO) and a patient name (PNT NAME). The report management page of FIG. 6 shows the page information produced using acknowledge flags and explanatory flags designated by a user at the upper portion thereof, i.e. a list of patients indicating an unacknowledged status of assessment reports for the execution results of medical examinations and patients having no explanation for the execution results of medical examinations based on assessment reports among all the patients to be examined according to the today's medical examination schedule. Alternatively, the report management page of FIG. 6 may show the page information produced using acknowledge flags and explanatory flags designated by a user at the lower part thereof, i.e. a list of patients indicating an acknowledged status of assessment reports for the execution results of medical examinations and patients having explanation for the execution results of medical examinations.

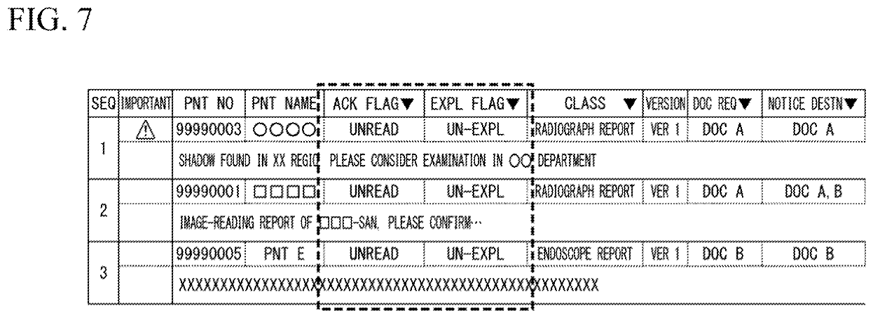

FIG. 7 shows a part of the report management page shown in FIG. 6, highlighting the columns of ACK FLAG and EXPL FLAG using dotted lines. Specifically, FIG. 7 shows the page information produced using acknowledge flags and explanatory flags designated by a user, enlarging a list of patients indicating an unacknowledged status of assessment reports for the execution results of medical examinations and patients having no explanation for the execution results of medical examinations based on assessment reports among all the patients to be examined according to the today's medical examination schedule. As shown in the columns encompassed by dotted lines, the report management part 13 produces the report management page describing the information of the patients assigned important flags, the patients linked with acknowledge flags indicating “unacknowledged” (UNREAD), and the patients linked with explanatory flags indicating “unexplained” (UN-EXPL).

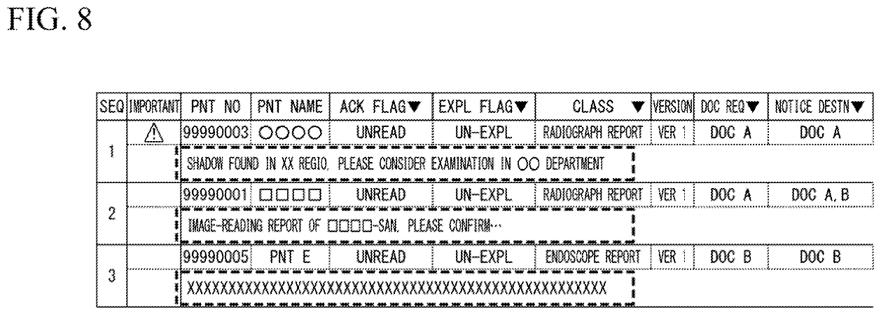

FIG. 8 shows a part of the report management page shown in FIG. 6, highlighting the comments in SEQ 1-3 using dotted lines. That is, the report management page may show the comments about the patients registered by the doctors making acknowledge requests of assessment reports.

The aforementioned process is explained in such a manner that both a top page of an electronic medical record and a report management page, which are concurrently transmitted by the patient management apparatus 1, are displayed in parallel on a monitor of the first terminal 3. When a patient is designated on an electronic medical record, the patient management apparatus 1 may transmit a medical record page of the designated patient to the first terminal 3 together with the report management page described above. For example, a user (or a doctor) may select a patient subjected to a medical examination on the top page of an electronic medical record when examining the patient. When the patient selected on the electronic medical record has not acknowledged an assessment report for the execution result of a medical examination or when the selected patient has no explanation for the assessment report, the report management part 13 of the patient management apparatus 1 may transmit to the first terminal 3 the report management page describing records such as an acknowledge flag, an explanatory flag, an examination type, the version of an assessment report, the name of a doctor making a request, the name of a doctor serving as a notice destination, an examination date, the date to produce the assessment report, the name of a doctor making a request to confirm the assessment report, a department of medical request assigned to the doctor, a last person to update those records, a last date to update those records, a doctor originating the request, and a comment made by the doctor producing the assessment report in connection with a patient number and a patient name.

When the report management page is displayed on a monitor of the first terminal 3, a user (e.g. a doctor) may check an acknowledge flag of the report management page. When the acknowledge flag indicates “unacknowledged” (UNREAD), the user confirms an assessment report of a patient. For example, the user inputs a patient number to the second terminal 4 communicating with the examination management apparatus 2 configured to transmit the assessment report. The second terminal 4 transmits a transmission request to transmit the assessment report to the examination management apparatus 2, and therefore the second terminal 4 receives the assessment report from the examination management apparatus 2. The second terminal outputs the assessment report with its monitor. In this connection, the second terminal 4 may output a radiographic image representing an example of the execution result of a medical examination. In addition, the user may check an explanatory flag of the report management page. When the explanatory flag indicates “unexplained”(UN-EXPL), the user may explain to a patient of the corresponding record a condition of the patient based on the assessment report, the result of observing the radiographic image, and user's own opinion, and then the user will operate the first terminal 3 to change the explanatory flag of the patient's electronic medical record from “unexplained” to “explained”. The first terminal 3 transmits to the patient management apparatus 1 an update request to request updating the explanatory flag from “unexplained” to “explained”, which includes at least a doctor ID, a patient number, and an examination number. Accordingly, the electronic medical record part 12 of the patient management apparatus 1 changes the explanatory flag from “unexplained” to “explained” in connection with a combination of the doctor ID, the patient number, and the examination number. This makes it possible for the patient management apparatus 1 to record an evidence indicating that the execution result of a medical examination has been explained to the patient.

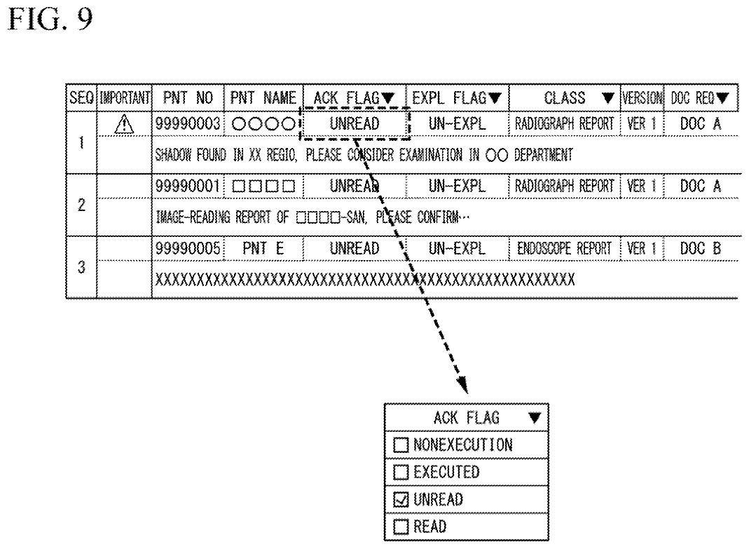

FIG. 9 shows a part of the report management page of FIG. 6, highlighting “UNREAD” in the column of ACK FLAG using dotted lines. After an acknowledgement of an assessment report, a user may change an acknowledge flag from “unacknowledged” (UNREAD) to “acknowledged” (READ). FIG. 9 shows an example of a screen image used to change the acknowledge flag. In FIG. 9, “NONEXECUTION” indicates that no medical examination has been executed while “EXECUTED” indicates that a medical examination has been completed. A user may explain to the patient the condition of the patient based on the assessment report, the result of observing a radiographic image, and the user's opinion, and then the user may operate the first terminal 3 to change the explanatory flag on the patient's electronic medical record from “unexplained” (UN-EXPL) to “explained” (EXPLAINED).

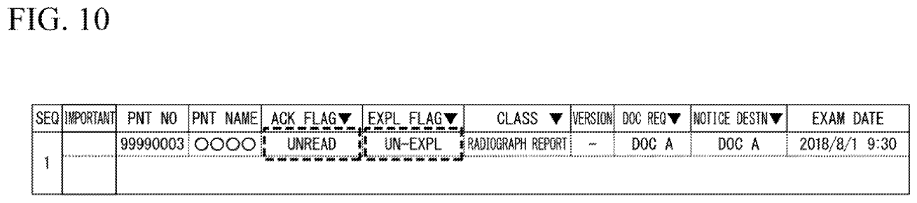

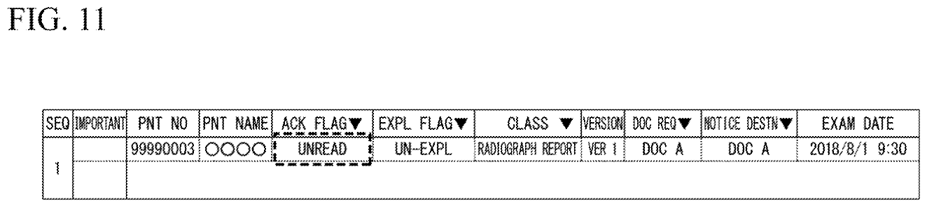

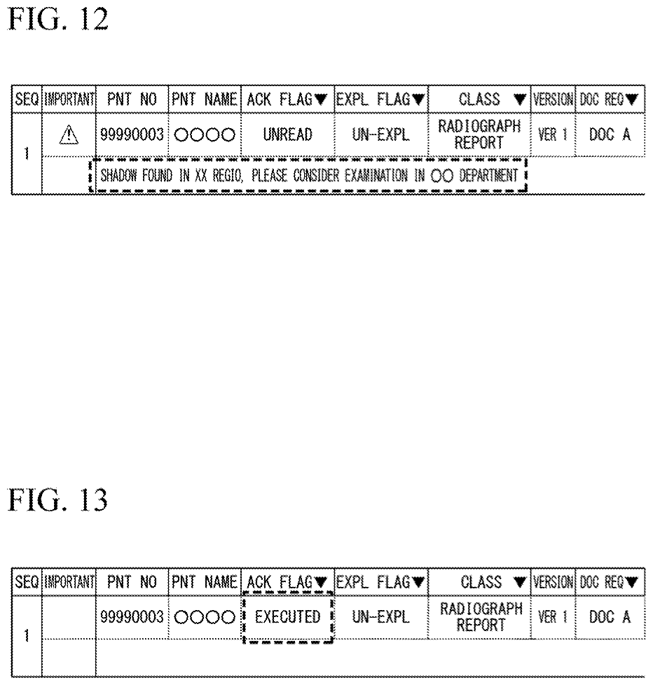

FIG. 10 shows a part of the report management page of FIG. 6, highlighting the columns of ACK FLAG and EXPL FLAG using dotted lines. FIG. 11 shows a part of the report management page of FIG. 6, highlighting the column of ACK FLAG using dotted lines. FIG. 12 shows a part of the report management page of FIG. 6, highlighting the comment in SEQ 1 using dotted lines. FIG. 13 shows a part of the report management page of FIG. 6, highlighting the column of ACK FLAG using dotted lines.

Next, user operations using the report management page will be further described in detail with reference to FIG. 10 through FIG. 13.

When a radiographic image is produced by executing a medical examination but an image-reading doctor has not acknowledged the radiographic image, the first terminal 3 may receive from the patient management apparatus 1 the report management page describing an execution flag indicating “executed”, an acknowledge flag being unset, and an explanatory flag indicating “unexplained”. In this case, the first terminal 3 displays on its monitor the report management page of FIG. 10 describing the acknowledge flag being set to “executed” and the explanatory flag being set to “unexplained”. Since the acknowledge flag is set to “executed” while the explanatory flag is set to “unexplained” on the report management page, a user (e.g. a doctor) may recognize that a medical examination has been completed but that an image-reading doctor has not confirmed a radiographic image while an assessment report has not been produced.

Thereafter, an image-reading doctor may produce an assessment report upon confirming a radiographic image. Subsequently, the image-reading doctor registers the assessment report and a completion of producing the assessment report in the examination management apparatus 2. At this time, the image-reading doctor designates an examination number and a patient number using the interface of the examination management apparatus 2. The examination management apparatus 2 transmits to the patient management apparatus 1 the production-completion information representative of a completion of producing the assessment report describing the examination number, the patient number, and a completion of registering the assessment report. In this connection, the image-reading doctor may register the comment describing any points which the image-reading doctor has been concerned with when producing the assessment report. Subsequently, the image-reading doctor will register the comment in the examination management apparatus 2. In this case, the production-completion information includes the text information representative of the comment.

The report management part 13 of the patient management apparatus 1 determines the record of the report management page using an examination number and a patient number so as to change an execution flag from “unexecuted” to “executed”. When the production-completion information includes the text information of the comment, the report management part 13 writes the text information into a comment field in the record of the report management page. Upon detecting a transmission request of the report management page, the report management part 13 of the patient management apparatus 1 transmits to the first terminal 3 the report management page describing an execution flag “executed”, an acknowledge flag “unacknowledged”, and an explanatory flag “unexplained” as well as the text information of the comment.

Due to a change of the acknowledge flag from “unset” to “unacknowledged”, the first terminal 3 displays on its monitor the report management page of FIG. 11 in which the acknowledge flag is updated with “unacknowledged” (UNREAD). In addition, the first terminal 3 displays on its monitor the report management page of FIG. 12 in which the text information of the comment is described in the comment field. Since the acknowledge flag is set to “unacknowledged” (UNREAD) while the explanatory flag is set to “unexplained” on the report management page, a user may recognize that a medical examination has been completed and that an image-reading doctor has confirmed a radiographic image and completed in producing an assessment report. In addition, the user may recognize the timing necessary to confirm the assessment report and to make an explanation for a patient. Moreover, the user may confirm the comment to take any measures based on the comment of an image-reading doctor.

The report management page may have a function for a documenter of an assessment report (e.g. an image-reading doctor) to notify a completion of producing an assessment report, e.g. “EXECUTED” in FIG. 13. For example, the image-reading doctor may operate a terminal communicating with the patient management apparatus 1 to input a patient number and an examination number, thus displaying the report management table on the terminal. In this case, the image-reading doctor may use a function of changing an acknowledge flag of the report management page to change the acknowledge flag with “EXECUTED”. The terminal transmits to the patient management apparatus 1 a record-update request describing the patient number, the examination number, and the acknowledge flag “EXECUTED”. The report management part 13 of the patient management apparatus 1 updates the acknowledge flag, which is recorded on the report management page in connection with the patient number and the examination number, with “EXECUTED”. Accordingly, the first terminal 3 may displays the report management page describing an acknowledge flag which is set to “EXECUTED” with respect to a medical examination of a patient.

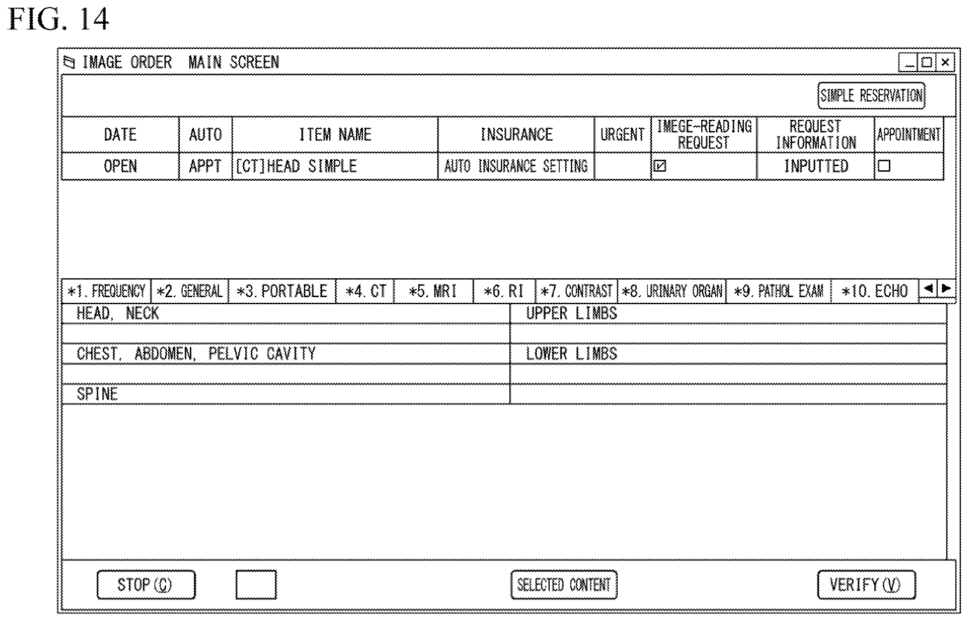

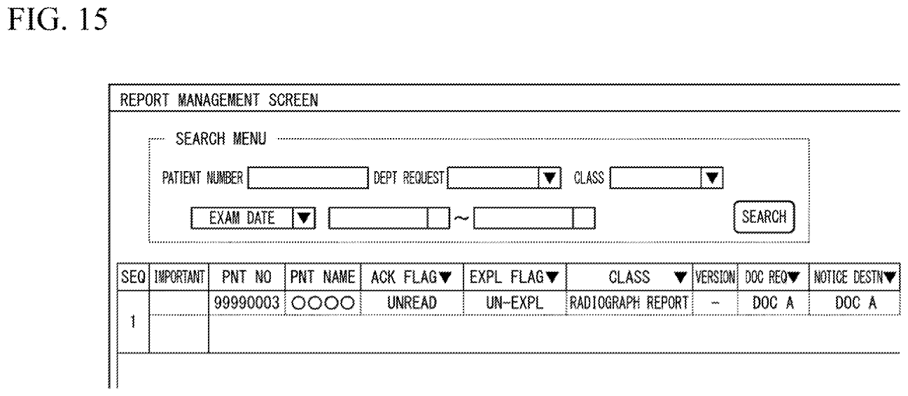

FIG. 14 shows an example of an examination request registration page. FIG. 15 shows a part of the report management page shown in FIG. 6. A user (e.g. a doctor) may register an examination request on the page of an electronic medical record. For example, the user may press an examination request registration button on the page of an electronic medical record dedicated to a predetermined patient. Upon detecting the pressing of the examination request registration button, the electronic medical record part 12 transmits an examination request registration page of FIG. 14 to the first terminal 3. The user may register various pieces of information such as an examination date, the name of an examination item, an examination type, and the like on the examination request registration page, and then the user may press an enter button. Subsequently, the first terminal 3 transmits to the patient management apparatus 1 an examination request including various pieces of information which are selected or input by the user on the examination request registration page. The patient management apparatus 1 registers various pieces of information included in the examination request in an examination request table of the database 104. An examination device or the examination management apparatus 2 acquires the information recorded on the examination request table so as to carry out a medical examination. According to the examination request, the electronic medical record part 12 outputs to the report management part 13 a record addition instruction to add records to the report management table. The record addition instruction includes a doctor ID (indicating the user who makes a record addition instruction to the report management table), a patient number, a patient name, an examination type, an examination number, and the like. As shown in FIG. 15, the report management part 13 adds to the report management page a single record including various pieces of information linked together.

Thereafter, as described above, a series of processes for a medical examination made by a clinical technician, and an assessment report for the execution result of a medical examination made by an image-reading doctor. In this case, the image-reading doctor may update the assessment report with respect to a radiographic image, which is another aspect of the execution result of a medical examination. Accordingly, it is possible to manage the version of an assessment report which may be changed in an order of a first version, a second version, a third version, and so on.

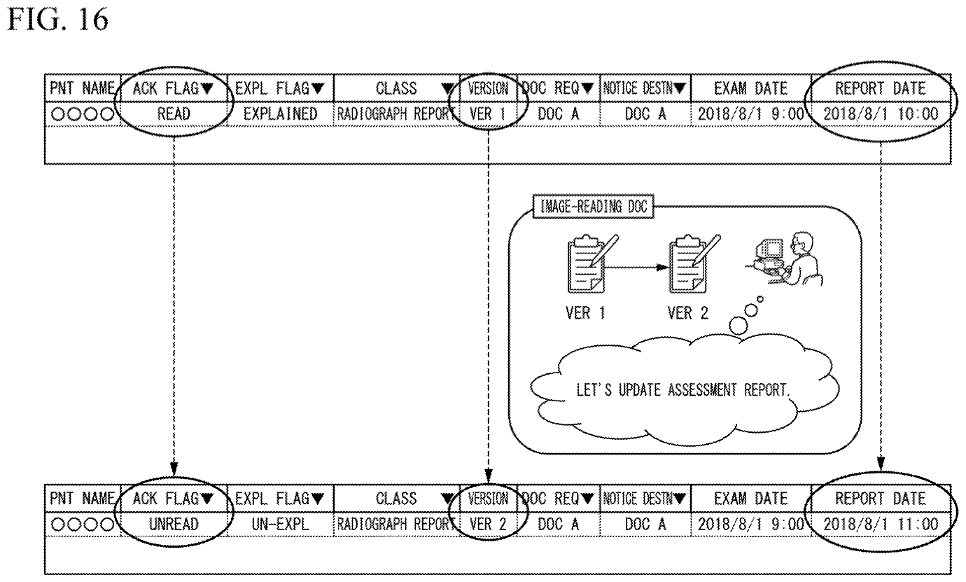

FIG. 16 shows a part of the report management table shown in FIG. 6, one record of which would be updated in columns. It is assumed that an image-reading doctor may newly produce a second version of an assessment report with respect to a medical examination, the execution result of which has been summarized as a first version of an assessment report, thus registering the second version of an assessment report. Herein, the second version corresponds to the latest version of an assessment report, which is produced by applying additions or modifications to the first version of an assessment report. At this time, the user may designate an examination number and a patient number via the interface of the examination management apparatus 2. The examination management apparatus 2 transmits to the patient management apparatus 1 the production-completion information including various pieces of information such as an examination number, a patient number, and a completion of producing an assessment report. The report management part 13 of the patient management apparatus 1 determines a record of the report management table using an examination number and a patient number. When the record has already registered the version of an assessment report, the report management part 13 increments the value of the version to set a new version in the record. When a acknowledge flag of the record of the report management table has been already set to “READ”, the report management part 13 updates the acknowledge flag with “UNREAD”. In addition, the report management part 13 updates the explanatory flag from “EXPLAINED” to “UNEXPLAINED (UN-EXPL)”. Moreover, the report management part 13 updates the date of producing an assessment report with the date to produce the second version of an assessment report.

According to the aforementioned process of FIG. 16, the patient management apparatus 1 may update an acknowledge flag with a value indicating that a user has not confirmed an assessment report after obtaining the version information of the assessment report when a previous acknowledge flag, which is stored in connection with a previous assessment report before updating, indicates “acknowledged”.

A reading request to read a radiographic image or an assessment report representing the execution result of a predetermined medical examination can be sent to one or more doctors. For this reason, the examination management apparatus 2 may accept registration of destinations to send a reading request. The destinations serving as doctors may include at least a doctor who makes an examination request, but the examination management apparatus 2 may accept registration of destinations as doctors having arbitrary attributes such as attending doctors in charge of patients in a hospital ward. The examination management apparatus 2 transmits to the patient management apparatus 1 the production-completion information describing an examination number, a patient number, a completion of registering an assessment report, and a doctor as a destination. The report management part 13 of the patient management apparatus 1 determines a record of a report management table using an examination number and a patient number. The report management part 13 registers a destination doctor included in the production-completion information as a destination doctor described in the record of the report management table. Accordingly, the report management part 13 transmits a report management page to a destination doctor when a plurality of doctors operates an electronic medical record.

Upon confirming an assessment report or after explaining an assessment report to a patient, a user (e.g. a doctor) may register a record with respect to the patient. For example, the record may include its corresponding content such as the SOAP type described above. The first terminal 3 transmits to the patient management apparatus 1 a registration request describing a patient number, an examination number, a content, and the like. The report management part 13 registers the content included in the registration request in a predetermined filed of a record linked to the patient number and the examination number.

The user may convert the record of the report management table into the text information in the CSV (Comma-Separated Values) format, which is downloadable into the first terminal 3. For example, the user may input an output parameter such as a patient number and a doctor ID into a search field of a report management page, thus displaying the desired information of the report management page. Then, the user may press a CSV-output button on the report management page. According to a CSV-output request and an output parameter designated by the user, the report management part 13 of the patient management apparatus 1 extracts the desired information of the report management page and converts it into the text information of the CSV format. The report management part 13 transmits the text information to the first terminal 3.

In this connection, the report management part 13 of the patient management apparatus 1 may have another function to display a list of users subjected to today's medical examinations.

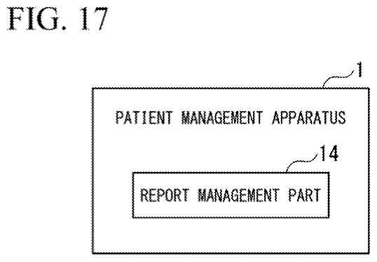

FIG. 17 is a block diagram showing a minimum configuration of the patient management apparatus 1. As shown in FIG. 17, the patient management apparatus 1 includes at least the function of the report management part 13. The report management part 13 is configured to obtain the execution result of a medical examination for a patient according to an execution request so as to record the execution result in connection with an acknowledge flag indicating whether or not to acknowledge the execution result and an explanatory flag indicating whether or not to make an explanation of the execution result. The report management part 13 determines a patient to be examined according to the today's medical examination schedule in connection with a patient number. The report management part 13 outputs a list of reading requests to read acknowledge flags, explanatory flags, and execution results linked to the identifications of patients to be examined according to the today's medical schedule.

Upon displaying the top page of an electronic medical record or a medical record page of a predetermined patient in an electronic medical record, the report management part 13 of the patient management apparatus 1 transmits to a user's terminal a patient management page describing a list of reading requests to read execution results linked to patient numbers of patients other than patients indicating acknowledge flags as “acknowledged” and explanatory flags as “explained” among all the patients to be examined according to the today's medical examination schedule. This makes it possible for a user (e.g. a doctor) to reduce a possibility to overlook an assessment report (i.e. an example of the execution result of a medical examination) and radiographic images, and therefore it is possible to improve an accuracy of explaining to patients their conditions based on execution results of medical examinations.

The aforementioned apparatuses such the patient management apparatus and the examination management apparatus include computer systems such that the foregoing processes are converted into programs and stored on computer-readable storage media, and therefore computers may load and execute programs to achieve the foregoing processes. Herein, computer-readable storage media may refer to magnetic disks, magneto-optical disks, CD-ROM, DVD-ROM, semiconductor memory, or the like. In addition, computer programs can be delivered to computers through communication lines, and therefore computers may execute programs to achieve the foregoing functions.

In addition, the present embodiment relates to a patient management apparatus and a patient management method; but this is not a restriction. For example, the present invention is applicable to any information processing apparatuses handling professional reports (e.g. medical reports or technological reports) which may be updated, checked, and explained to related persons by professionals.

Lastly, the present invention is not necessarily limited to the foregoing embodiment which can be modified in various ways within the scope of the invention as defined in the appended claims, and therefore the present embodiment may include any variations, modifications, and design changes without departing from the subject matter of the invention.

What is claimed is:

1. A patient management apparatus comprising:
an electronic medical record part configured to acquire and record execution results of medical examinations for patients in connection with identifications of patients, acknowledge flags indicating whether the execution results have been acknowledged, and explanatory flags indicating whether the execution results have been explained to patients; and
a report management part configured to determine patients to be examined according to a today's medical examination schedule in connection with the identifications of the patients and to thereby output a list of reading requests to read the execution results, the acknowledge flags, and the explanatory flags in connection with the identifications of the patients to be examined according to the today's medical examination schedule.

2. The patient management apparatus according to claim 1, wherein the execution results of medical examinations for the patients are each configured of an assessment report obtained from an examination management apparatus configured to manage medical-examination information, wherein the electronic medical record part is configured to record the assessment report in connection with an acknowledge flag indicating an unacknowledged status of the assessment report and an explanatory flag indicating no explanation provided to the patient with respect to the assessment report, wherein according to a user operation made by a user confirming the assessment report, the report management part is configured to update the acknowledge flag to indicate an acknowledged status of the assessment report and to update the explanatory flag to indicate the patient having an explanation of the assessment report.

3. The patient management apparatus according to claim 2, wherein the report management part is configured to automatically produce the list of the reading requests to read the execution results of the medical examinations according to a user operation to start processing.

4. The patient management apparatus according to claim 2, wherein upon obtaining information representing an updated version of the assessment report, the report management part is configured to update the acknowledge flag to indicate the unacknowledged status of the assessment report when the acknowledge flag indicates the acknowledged status of the assessment report is recorded in connection with a previous version of the assessment report.

5. The patient management part according to claim 2, wherein upon obtaining information representing an updated version of the assessment report, the report management part is configured to updated the explanatory flag to indicate no explanation provided to the patient with respect to the assessment report when the explanatory flag indicating the explanation provided to the patient is recorded in connection with a previous version of the assessment report.

6. The patient management apparatus according to claim 2, wherein the report management part is configured to apply an important flag to an execution result selected from among the execution results of the medical examinations within the list of reading requests according to the user operation.

7. A patient management method comprising:
acquiring and recording execution results of medical examinations for patients in connection with identifications of patients, acknowledge flags indicating whether the execution results have been acknowledged, and explanatory flags indicating whether the execution results have been explained to patients;
determining patients to be examined according to a today's medical examination schedule in connection with the identifications of the patients; and
outputting a list of reading requests to read the execution results, the acknowledge flags, and the explanatory flags in connection with the identifications of the patients to be examined according to the today's medical examination schedule.

8. A computer-readable storage medium having stored therein a program causing a computer to implement a patient management method comprising:
acquiring and recording execution results of medical examinations for patients in connection with identifications of patients, acknowledge flags indicating whether the execution results have been acknowledged, and explanatory flags indicating whether the execution results have been explained to patients;
determining patients to be examined according to a today's medical examination schedule in connection with the identifications of the patients; and
outputting a list of reading requests to read the execution results, the acknowledge flags, and the explanatory flags in connection with the identifications of the patients to be examined according to the today's medical examination schedule.
